(19) United States
(12) Patent Application Publication
(10) Pub. No.: US 2005/0287536 A1
(43) Pub. Date: Dec. 29, 2005

(54) Disposable, multi-use, DNA sample collection disk apparatus for field biohazard testing, DNA testing, and personal authentication

(76) Inventor: Douglas Kozlay, Timonium, MD (US)

Correspondence Address: Douglas Kozlay, Suite 420, 9475 Deereco Road, Timonium, MD, 21093, US

(21) Appl. No.: 10/876,819

(22) Filed: Jun. 24, 2004

(51) Int. Cl. C12Q 1/68; C12M 1/34

(52) U.S. Cl. 435/6; 435/287.2

(57) ABSTRACT

An easy-to-use, versatile, disposable, customer-replaceable, multi-use, DNA sample collection disk apparatus for use with fixed, portable, or field-based DNA sample collection, analysis, and detection systems is disclosed. General features of the invention are its' small form factor, portability, wearability, ease-of-use, and self-contained capacity to collect and analyze multiple different human DNA samples and sample types. The special utility of the invention is demonstrated in the field wherein neither trained medical personnel, nor conventional DNA testing labs, are necessary to operate the invention. Invention preferred embodiments include multipurpose cards or badges designed to (1) electronically authenticate subjects identities using DNA samples, and/or electronically detect presence or absence of biological agents (e.g., anthrax) and/or chemical agents (e.g., Sarin) using DNA samples; and (2) perform other DNA-based, protein-based, or other analytic and/or identification functions.

### FIELD OF THE INVENTION

The fields of the invention encompass DNA-based biological, chemical, and disease agent detection, and DNA-based personal authentication, using DNA samples. Testing detects absence or presence of selected “target DNA” when compared to “reference DNA”. If a human is exposed to biological, chemical, or disease agents, their human DNA sample will reflect this fact with high statistical probability. For my primary intended applications—portable, easy-to-use, field-based DNA testing—the best way to implement the invention is to use “card-like” or “badge-like” platform vehicle(s).

### NOTICE REGARDING COPYRIGHTED MATERIAL

A portion of the disclosure of the patent document contains material which is subject to copyright protection. The copyright owner has no objection to the facsimile reproduction by any one of the patent document or the patent disclosure as it appears in the Patent and Trademarks Office file or records, but otherwise reserves all copyright rights whatsoever.

### BACKGROUND OF THE INVENTION

Notwithstanding that DNA testing is well known in the art, present markets demand an easy-to-use, versatile, customer replaceable, multi-use, DNA sample collection disk apparatus for use with fixed, portable, or field-based DNA sample collection, analysis, and detection systems. Ordinarily, “conventional” DNA testing requires access to trained technicians and/or medical personnel, as well as access to a laboratory and necessary equipment. These techniques often require the use of radioisotope or fluorescent labels as well as expensive post-hybridization reagent processing steps. Eliminating most of the expensive reagents and labor involved in the labeling of DNA will thereby significantly reduce time, effort and expense. Existing electrochemical techniques require more extensive plug-in boards or modules and/or analysis machinery that must be cleaned or replaced after each sample.

The invention uses a handheld or wearable device that externally appears to be a high-impact plastic (or other durable material) badge, portable device, or smartcard. For personal authentication applications, the invention can self-authenticate user subject(s) after submission of a DNA sample (e.g., a skin scraping), using predetermined reference DNA. The authenticating user places a sample on a sample collection well on one sample segment of a removable, disposable disk. The pre programmed processor and electronic circuitry aboard the unit to which the disk is attached match the submitted DNA sample (the “sample analyte”) with stored reference DNA and determine whether or not there is a match. Further, the device can be programmed to provide a display, an audible alarm, and/or transmit an alert message, based on the testing outcome. In addition to authenticating a user, the platform can be configured to test for the presence or absence of disease agents, biological agents, or chemical agents inherent within the DNA sample of subjects who submit samples for analysis.

### RELATED ART

Recently, patent application 20030086591 to Simon, entitled “Identity Card and Tracking System,” described a card that employs biometric data, including “DNA fingerprints”, to identify individuals by comparing the data stored on a card with a reference set of data in a central registry. Notwithstanding, there is increasing and significant controversy surrounding the many privacy risks of authentication devices which must interact with central systems. Such central-registry-oriented products, when compromised, allow privacy invasion by hackers and other unauthorized users. This application also is silent on the topic of removable, disposable sample collection disks. Also, the present invention respects privacy by not being configured to interact with a central registry, unless explicitly so configured. Further, the present invention is oriented toward portable, easy-to-use, self-contained testing.

U.S. Pat. No. 6,376,177 to Poponin describes a spectroscopic system for detecting molecular hybridization by means of a system comprising the following items: a near-field Surface-Enhanced Raman Scattering (SERS) substrate arranged to support one or more predetermined hybridizable molecules, a coherent radiation source arranged to impinge the radiation onto each of the hybridizable molecules, Raman spectrograph arranged in a photon receiving relationship to the photonic collector, and electronics to receive the output of the Raman spectrograph and to convert it to an electronic output indicative of the presence or absence of hybridized molecules on the SERS substrate. This method enables the detection of hybridization on as little as one molecule quickly and with higher accuracy than current electrochemical methods. Although the Poponin patent is useful, it is silent on the topic of removable, disposable sample collection disks and portable, easy-to-use, self-contained testing.

U.S. Pat. No. 6,606,157 to Kaye discloses a fiber detector assembly comprising: (i) a scattering chamber body; (ii) means for drawing airborne particles through a chamber adapted so particles travel “single file” with longitudinal axis of particles with elongate shape, substantially aligned with the direction of the air flow; (iii) means for illuminating the particle stream within the chamber body; (iv) an optical detector adapted to intercept and collect a portion of light scattered by particles passing through the illuminating beam; (v) data processing means adapted to capture and process signals from the optical detector, where the optical detector comprises a photodiode array consisting of a central opaque area surrounded by two or more annular rings of detector elements. The cited invention is essentially oriented to detect airborne fibers of asbestos, but is not oriented to do DNA-based authentication and/or DNA testing, as in the present invention. The most abundant asbestos mineral, white asbestos is present in 95% of contaminated installations. The second most commonly found variety is blue asbestos, with brown asbestos being a third type of rare form. All three materials produce fibers that can penetrate deep into lungs and because of their shape eventually become entrapped there. Unlike the present invention disclosed herein, Kaye's patent is also silent on the topic of removable, disposable sample collection disks and portable, easy-to-use, self-contained testing.

By further contrast, the present invention greatly simplifies the identification process by utilizing DNA signatures to extract DNA samples and perform the entire identification process on the card eliminating the need for a laboratory and a separate sample preparation step. Importantly, the invention disclosed herein also requires no “cleaning”—because of its' “disposable”, customer-replaceable sample collection disk where the “full” sample collection disk is merely removed and replaced with a fresh, unused collection disk—it is not subject to the expensive and time-consuming cleaning and subsequent inaccuracies.

U.S. Pat. No. 5,041,203 to Serwer, describes an apparatus and a procedure for fractionating DNA using agarose gel electrophoresis. To improve resolution by length and conformation, the direction of the electric field impressed upon the sample is changed by rotating the gel. Similarly Tomblin in U.S. Pat. Nos. 4,750,982 and 4,617,102 disclose a laboratory apparatus to concentrate DNA in an agarose gel disc. Both the Serwer and Tomblin patents refer to a complex laboratory apparatus for electrophoresis using agarose gels, as opposed to the simple portable device using ferrocene technology requiring no laboratory to directly detect DNA materials in the manner of the present invention.

Infineon® Technologies has also reportedly developed a “fully electronic DNA Sensor with 128 positions” and “in-pixel A/D conversion”. Infineon claims to have developed this approach to DNA sample detection involving multiple sensor “pixels”. Each sensor-“pixel” contains a circuit that controls the sensor electrode voltages and provides amplified copies of the sensor currents at the pixel output.

While this product would appear to be utilitarian as advertised, it would appear to be an expensive way to analyze targets, given cost of replacing active electronics such as amplifiers and Analog to Digital (A/D) converter chips. Infineon also reports that experience to date with such devices indicates about a 20% drop in sensitivity during cleaning, even when washed under laboratory conditions. This would prevent indefinite use of a single device (i.e., beyond several uses). Although the Infineon product appears useful, it is however silent on the topic of removable, disposable sample collection disks and portable, easy-to-use, self-contained testing, unlike the present invention.

All the related art that has been cited herein represents a significant amount of technological progress, however, none of the above-related art can compare equally with the present invention.

### NECESSITY OF THE INVENTION

Current apparatuses in the market and methods used for DNA testing require the use of trained medical personnel and use expensive cards, modules, and instrumentation for sample processing. The alternative to existing sample cards, modules, instrumentation, etc., is to use “permanent” sample processing equipment that must be cleaned after each use. This adds expense, decreases accuracy, and makes it difficult to provide equipment that can be worn or carried by a user. What is needed in the art is a device that is as easy for untrained personnel to use, as a typical “disposable” pocket camera.

### OBJECTS OF THE INVENTION

Accordingly, one primary object of my invention is to provide an apparatus, method, and system with easy-to-use, self-contained, “onboard” capability to handle multiple DNA sample collection and analysis events.

Another object of the invention is to enable the use of untrained personnel in the field to repetitively test for, and detect the presence or absence of chemical agents, biological agents, and disease agents that can be found within DNA sample analytes, when compared with predetermined reference DNA samples.

Another primary object of the invention is to provide a reliable, multi-functional user authentication apparatus, method, and system with multiple testing and self-authentication modalities.

Yet another primary object of the invention is to provide a convenient, relatively economical means for subsequent verification of test results, forensic sample preservation, and archival storage.

### SUMMARY OF THE INVENTION

The present invention describes an easy-to-use, stand-alone, self-contained electronic sample collection disk apparatus. The disk apparatus contains sample segments. Each sample segment contains small packets of probe reagent solution; DNA “sample collection wells”, for sample submission (e.g., skin, mucous, saliva etc. samples); and one or more electronic DNA probes with single-strand DNA reference samples. A microprocessor is also embedded on the testing platform apparatus with appropriate comparator circuitry and components including a power source. The microprocessor and circuitry interfaces with each sample well in succession, to determine if a match has taken place. Optionally, depending on apparatus configuration(s), the test results can be displayed on the testing platform apparatus unit itself, and/or results can be transmitted to another destination, and/or the test apparatus can be programmed to provide a local audible alarm and/or a visual cue, indicating a match.

This invention can be implemented using either of two basic detection methods, electrochemical or spectrographic. One such electrochemical method is taught by Bamdad in U.S. Pat. No. 6,541,617 which describes detection using a “sandwich hybridization assay” wherein three critical components (capture probe, target, and signaling probe) are each present. Electrons flow to the electrode surface only when the target is present and specifically hybridized to both signaling and capture probes. The current generated by this system is converted to digital data and interpreted by a computer to determine the presence or absence of hybridization. The other spectrographic method, taught by Poponen in U.S. Pat. No. 6,376,177, describes a spectroscopic system for detecting molecular hybridization by means of a near-field Surface-Enhanced Raman Scattering (SERS) substrate arranged to support predetermined hybridizable molecules, a coherent radiation source arranged to impinge the radiation onto each of the hybridizable molecules, a Raman spectrograph arranged in a photon receiving relationship to the photonic collector, and with resultant output conversion to digital data and interpretation by a computer to determine the presence or absence of hybridization.

The invention as described hereinafter can be implemented using either of these methods or similar methods of detecting hybridization.

The methods described by Bamdad and Poponin do not use the preexisting fluorescent marker technologies that are commonly used in DNA laboratories. The disposable customer replaceable sample collection disk of the present invention is referred to as “non-fluorescent” to distinguish it from so-called “optical bio-disk” technology that is the basis of U.S. Patent Application 20020168652, “Surface assembly for immobilizing DNA Capture Probes and bead-based assay including optical bio-discs and methods relating thereto.”

The entire invention is essentially embedded/packed as a whole encompassing unit in a “badge-like” platform similar to that employed upon a (usually plastic-base) substrate. The present invention, basically, is “much smarter than” a typical badge, given its' effective functions as a “DNA sample testing laboratory that is handheld or wearable. The system of the invention is established on a thickened, “ruggedized” high-impact plastic case upon which is mounted a disposable, customer-replaceable, multi-use, DNA sample collection disk. This disk is capable of collecting and hybridizing samples in a compact and easy-to-use form that can be preserved for subsequent analysis or disposed of without requiring the subsequent cleaning of the sensor equipment. This permits the user of the device to index to the next successive sample segment as easily as advancing the film in a camera.

In addition to the disposable disk, the system contains at least one embedded “DNA sample-DNA reference comparator” microprocessor; a display readout or “status indicator”; a non-volatile memory; and a power source. The overall system is adapted for detecting and matching at least one target DNA sample (analyte) with at least one embedded DNA comparison reference sample.

### BRIEF DESCRIPTION OF THE DRAWINGS & REFERENCE NUMERALS

FIG. 1 Illustrates Protective Case and Cover, one Sampling Segment and Well, and Optional Display Window;

FIG. 2 Illustrates Disposable Sample Disk, Electronic Circuit with Microprocessor, and Battery Pack;

FIG. 3 Illustrates a close-up view of Disposable, Rotating, Circular-Shaped Sample Collection Disk, and Disk Mounting and Rotating Spindle;

FIG. 4 Illustrates a close-up view of Sampling Segment; and

FIG. 5 Ilustrates a Miniaturized Implementation of a Simplified Raman Spectrometer 100 Protective Case and Cover

### REFERENCE NUMERALS

102 Disposable Sample Disk
104 Optional Display Window
106 Electronic Circuit with Microprocessor
108 Battery
114 Disk Mounting and Rotating Spindle
200 Sampling Segment
202 Reagent Solution Packet
204 Solution Delivery (Conduit) Duct
206 Sample Well
207 Sample Collection Duct
208 Sample Delivery (Conduit) Duct
210 Electrode Substrates
211 Electrical Conductor
212 Electrical Contacts
300 Photon Collector
302 Half-Silvered Mirror
304 Prism
306 CCD Detector Array
308 Laser Diode and Collimating Lens
310 A/D Converter and Microprocessor

### DETAILED DESCRIPTION OF THE INVENTION

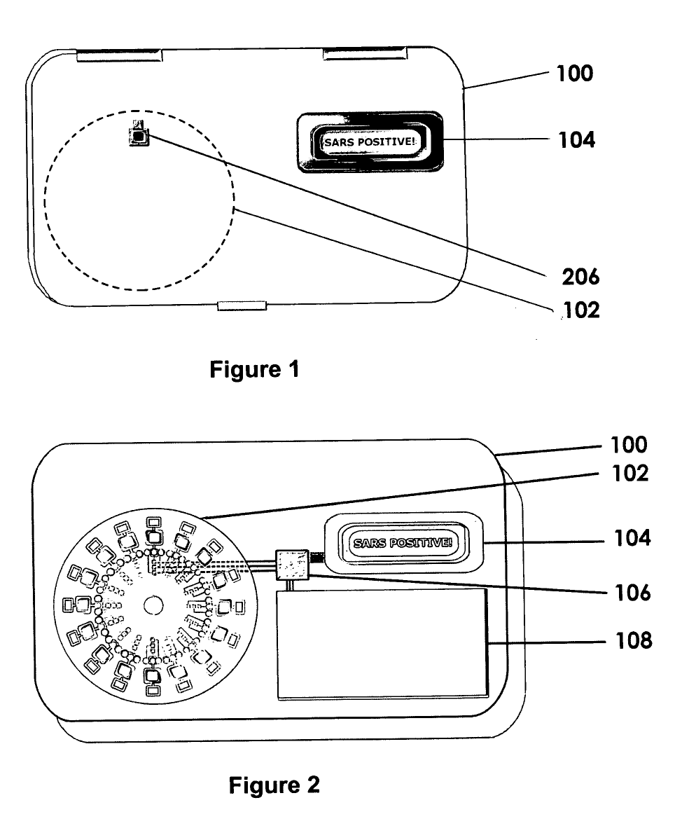

FIG. 1 is an exemplary illustration of a protective case and cover 100 that can be designed having various geometric shapes without interfering with substance of the invention. FIG. 1 also illustrates a “cover through” view and an exterior exposure of one section of one sampling segment 200 and sampling well 206. After closing the cover, the subject places his or her DNA sample (e.g., a skin scraping) through an aperture opening to one sampling segment and sampling well for testing. Additionally, optional display window 104 is illustrated to the right of the case and cover 100. The “results” of any sample testing indicating DNA match or exposure to any chemical agents accomplished by the invention are shown in window 104. Alternatively, if window 104 is not implemented, other indicators, such as visual or audible “cues” or combinations of visual, audible, and window 104 can be provided.

FIG. 2 illustrates the case 100 with the cover folded back, to allow better access to one of the most critical features of the invention—the disposable, rotating, customer-replaceable, multi-use DNA sample collection disk 102. FIG. 2 further shows the optional display window 104 is distinguished from disk 102 and phantom lines indicating the embedded, internal presence of an electronic circuit and embedded microprocessor 106. Battery Pack 108 provides direct current power to the electrical circuit, enabling the present invention to function as designed.

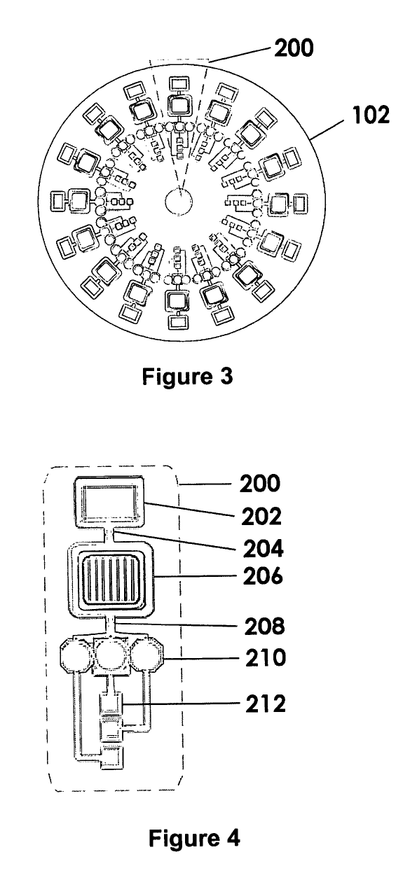

FIG. 3 shows a close-up view of disposable, rotating, circular-shaped sample collection disk 102, and disk mounting and rotating spindle 114. Sample collection disk 102 is affixed onto the device platform by means of the spindle 114. Accordingly, it should be noted that disk mounting and rotating spindle 114 is a critical feature of the invention, because once all the sample segments implemented on sample collection disk 102 are used up and/or the user decides to change disks—the user simply disengages the mounted disk 102 by pulling it off of the device platform from its deployed position on spindle 114, and the user then replaces disk 102 with a new and unused sample collection disk—it is readily observed, this facilitates and enables multiple sampling uses of all sample collection disks of the present invention. In one optional embodiment of sample collection disk 102, there can be implemented an onboard motorized function for either advancing rotation of disk 102 from one sampling segment to the next sequential sampling segment, (analogous to the “click and shoot” mechanism of a motorized film camera), or, selectively advancing rotation of disk 102 to a non-sequential sampling segment to select a different testing area on the sample disk (analogous to selecting a favorite song on a music CD or a specific chapter on a movie DVD). In operation, a sample collection disk such as disk 102 is positioned on the device platform, and generally, the disk is started at a first sampling segment position and is used. When use of the first position sampling segment is completed, the disk 102 is advanced to a second sampling segment position, and in turn to a third position and successive positions until either all sampling segment positions are used up or until the user elects to remove one sample collection disk and replace it with another sample collection disk (e.g., to change the type of testing being done, to change from one organization being tested to another organization, etc.).

FIG. 4 illustrates a close-up view of sampling segment 200. FIG. 4 illustrates a reagent solution packet 202. After the subject provides his or her DNA sample (e.g., a skin scraping) into sample well 206, the subject or the administering technician uses finger pressure to “rupture” (i.e., break open) solution packet 202 by pressing on a “rupture seal” or stopper (not shown) at the top of solution (conduit) duct 204. This conduit function duct 204 permits and channels the contents of packet 202 to flow into the sample well 206. Once the collected sample in sample well 206 (and sample well collection ducts 207) is mixed with the contents of packet 202, the mixed reagent and sample analyte is further channeled and ducted via sample delivery (conduit) ducts 208 onto electrode substrates 210. Electrical contacts 212 mate with power distribution contacts (not shown) extended from electronic circuit 106. FIG. 4 also shows the electrode substrate section 210 of sampling segment 200. While only three electrodes are depicted, any practical number of electrodes can be implemented therein in the invention. Where the ferrocene method of analysis is used, electrical conductor 211 allows electrode(s) 210 interconnection into electrical contact(s) 212.

Two possible primary preferred embodiments and operating methods employ electrochemical analysis techniques and spectrographic analysis techniques.

A first preferred operating method of the present invention uses the basic “ferrocene-based” complementary base pairing method. This method of operation uses chip-embedded DNA reference samples (electrically conductive ferrocene molecules) which are tethered to gold (Au) electrodes (or suitable equivalent electrode substrate). Electro-active compounds comprising DNA samples (analytes) are submitted by the test subject. If the submitted DNA test sample attaches to the reference sample containing ferrocene, a measurable electron transfer is detected. Gold (Au) electrodes are typically considered optimal.

A variation on the first preferred method of the present invention uses a “stem-loop-forming” piece of DNA tethered to a gold (Au) electrode, to which an electro-active ferrocene compound is attached in its' closed configuration. The DNA's hairpin structure keeps the iron-containing ferrocene near the electrode's surface, where it can undergo a measurable electron transfer with the electrode by means of electron tunneling.

Another variation of the first preferred method of the invention employs the EDDA (electrically detected displacement assay) method in which the capture probe DNA is not labeled and weakly-bound ferrocene-labeled signaling probes are removed from the capture probe by the DNA hybridization, thus decreasing the ferrocene electrochemical signal.

A second preferred embodiment of the invention employs a spectrographic method for detecting molecular hybridization by means of a near-field Surface-Enhanced Raman Scattering (SERS) substrate organized to support predetermined hybridizable molecules; a coherent radiation source arranged to impinge the radiation onto each of the hybridizable molecules; a Raman spectrograph arranged in a photon receiving relationship to the photonic collector; and with output conversion to digital data and interpretation by a microprocessor and associated circuitry means for determining the presence or absence of hybridization indicative of “match” or “no match”.

It is important to note, it is expected that the three electrochemical methods of the first preferred embodiment are well known to one skilled in the art. It is likewise expected, the methods of the second preferred embodiment are also well known to one skilled in the art.

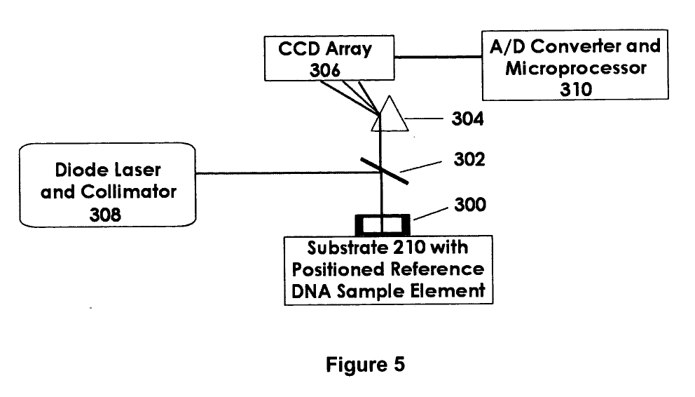

FIG. 5 describes a miniaturized implementation of a simplified Raman spectrometer of the second preferred embodiment suitable for the portable device of this invention. A monochromatic laser diode and collimator 308 provides collimated radiation to impinge on the sample at 210 via a half-silvered mirror 302. Fixed reference DNA sample molecule(s) are either pre-positioned during disk manufacture on electrode 210, or optionally can be positioned with a crystal micropositioner. A photon collector 300 returns the scattered photons via the half-silvered mirror 302 for spectral analysis. The prism 304 distributes the photons by frequency to the charge-coupled device (CCD) array detector 306. The CCD array signal is converted to digital data and is processed at the A/D converter and microprocessor 310. Thus, the Raman spectrometer system can be implemented with miniaturized optics and small semiconductor chips. In this embodiment, the electrode platform 210 is analyzed optically and there is no need for the electrical connections 212 of FIG. 4 except to provide grounding. This technique is also known to one skilled in the art of SERS.

In the foregoing specification, the invention has been described with reference to specific exemplary embodiments thereof. It will, however, be evident that various modifications and changes may be made thereunto without departing from broader spirit and scope of the invention as set forth in the appended claims. For example, various techniques can be used to implement the disclosed invention. Also, the specific logic presented to accomplish tasks within the present invention may be modified without departing from the scope of the invention. Many such changes or modifications will be readily apparent to one of ordinary skill in the art. The specification and drawings are, accordingly, to be regarded in an illustrative sense, the invention being limited only by the provided claims.

1. An easy-to-use DNA sample collection disk apparatus adapted for fixed, portable, and field-based DNA sample collection, analysis, and detection, comprising: a disposable, removable, customer-replaceable, circularly-shaped, rotating sample collection disk affixed onto a device platform by means of a disk mounting and rotating spindle.

2. The disposable, removable, customer-replaceable, circularly-shaped, rotating sample collection disk apparatus of claim 1, wherein said sample collection disk also includes a plurality of sample segments, each of which is independently adapted for collection of multiple sample analytes and multiple sample types, mixing a reagent solution with said sample analytes, and testing said sample analytes, and wherein said disk is further adapted for storage and archiving of sample analytes and test results.

3. The apparatus of claim 2, wherein each of said plurality of sample segments includes a sample collection well for collecting sample analytes, a packet containing a reagent solution for mixing with said sample analytes, a duct for ducting said reagent solution into said sample collection well, and at least one duct for ducting the reagent solution mixed with said analyte samples onto electrode substrates.

4. The apparatus of claim 2, wherein said apparatus is adapted for multiple-uses by means of rotating the disk from a first position allowing use of a first sample segment, to a second position allowing use of a second sample segment, and so forth in turn, such that each subsequent sample segment is presented in turn for use, until all such sample segments are used, at which time said disk is removed and replaced with a fresh disk.

5. An easy-to-use, self-contained, portable system for DNA sample collection and analysis, including a disposable, customer-replaceable, multi-use, rotating DNA sample collection disk apparatus affixed onto a platform device by means of a disk mounting and rotating spindle, at least one DNA sample detection method, a plurality of sample segments disposed upon said disk for collecting and analyzing sample analytes, an electronic circuit with a microprocessor for comparing DNA sample analytes with DNA reference samples, an optional display readout, and a power source enclosed in a ruggedized, portable case.

6. The system of claim 5, wherein said system is adapted for at least one of: (i) the detection, comparison, and matching of at least one DNA sample analyte with at least one DNA reference, and (ii) the DNA-based self-authentication of at least one predetermined authorized user.

7. A simple, easy-to-use method for repetitive DNA testing without trained medical personnel and without a DNA testing laboratory, including the steps of:
a. constructing the self-contained DNA testing system of the present invention including a disposable, removable, customer-replaceable, rotating, multiple use sample collection disk apparatus,
b. mounting said sample collection disk apparatus onto a disk mounting and rotating spindle,
c. positioning said disk apparatus to a first sample segment position for receiving DNA sample analytes until completion of first sample collection, then rotating said disk to the next desired sample segment position,
d. positioning said disk apparatus to a second sample segment position for receiving DNA sample analytes until completion of second sample collection, then rotating said disk again—to the next desired sample segment position,
e. positioning said disk apparatus in turn to any subsequent sample segment position(s) for receiving sample analytes until completion of sample collection,
f. removing said disk apparatus from said mounting and rotating spindle and replacing said disk apparatus with another disk apparatus, and
g. optionally storing and archiving said removed, replaced disk apparatus for further verification and analysis.

8. The system of claim 5, wherein the method for DNA sample analysis is by electrochemical means.

9. The system of claim 5, wherein the method for DNA sample analysis is by Raman spectrographic means.

10. The sampling segment of claim 3, wherein the sample well is adapted to scrape skin samples directly into the sample collection ducts.

11. The sample collection disk of claim 1, wherein said disk is motorized instead of manually positioned, to permit “click-and-shoot” sampling.

12. The motorized disk of claim 11, wherein said motorized disk can be automatically advanced to allow the collection of multiple samples and subsequent automated analysis.

13. The motorized disk of claim 12, wherein said motorized disk can be advanced sequentially.

14. The motorized disk of claim 12, wherein said motorized disk can be advanced from one predetermined location to another different predetermined location.
